(19) United States
(12) Patent Application Publication
(10) Pub. No.: US 2025/0302713 A1
(43) Pub. Date: Oct. 2, 2025

(54) Product and Method For Preparing of Cationic Nanoparticle Formulation For Dermal Treatment

(71) Applicant: TRUTEK CORP., Somerville, NJ (US)

(72) Inventors: Ashok Wahi, Basking Ridge, NJ (US); Kanika Wahi, Basking Ridge, NJ (US); Kevin Michael Bartolome, Manville, NJ (US); Tarun Kukreja, Hinsdale, IL (US); Abd Almonem Doolaanea, North Richland Hills, TX (US)

(21) Appl. No.: 19/236,153

(22) Filed: Jun. 12, 2025

(51) Int. Cl. A61K 8/41; A61K 8/02; A61K 8/37; A61K 8/73; A61K 8/92; A61Q 17/00

(52) U.S. Cl. CPC A61K 8/416; A61K 8/0241; A61K 8/37; A61K 8/73; A61K 8/731; A61K 8/735; A61K 8/922; A61Q 17/00; A61K 2800/95

(57) ABSTRACT

The present invention is a method of conditioning and treating a person's skin with a novel dermal care formulation characterized by the combination of (a) a cationic nanoparticle composition; (b) a film-forming ingredient; and (c) at least one cationic ingredient not encapsulated within the nanoparticle composition. The formulation may or may not contain a pharmacologically active ingredient. This three-fold system provides a comprehensive approach to dermal care, offering anti-aging, environmental protection, moisturizing and rejuvenating properties. As described and claimed herein, the invention consists of the method of conditioning and treating the skin, the formulations that are applied to skin, and methods of preparing those formulations.

### FIELD OF THE INVENTION

This invention is related to compositions of cationic nanoparticle formulation and methods of preparing them wherein the compositions are suitable for dermal applications like anti-aging, protecting the skin from environmental pollution, moisturizing and rejuvenating.

### BACKGROUND OF THE INVENTION

Your skin is your body's first shield against harmful things. Keeping it healthy is really important for everyone. Aging of the skin occurs normally over time. As we get older, our bodies make less collagen and elastin-proteins that keep skin firm and stretchy. The skin becomes thinner, drier, and wrinkled. However, environmental aging comes from outside sources such as (1) sun exposure (UV rays), (2) pollution, (3) smoking, and (4) poor diet and stress.

Many people use skin products every day to keep their skin healthy and to fight signs of aging. These products work in different ways. They can moisturize skin, keep it hydrated, remove dead skin cells, help new cells grow, form a thin protective layer, or shield skin cells from pollution.

Skin products often have special ingredients, like retinol or bakuchiol. These ingredients usually need to soak deep into your skin to work. But skin is tough to get through. So, cosmetic chemists have found different ways to help these ingredients get absorbed better. These ways include hydrating the skin, using ingredients that help things pass through, and using tiny particles called nanoparticles.

Nanoparticles are often used in skin products to carry these special ingredients deeper into the skin. For example, tiny fat bubbles called liposomes are known to get into the skin and deliver what's inside. Nanoparticles smaller than 10 nanometers can actually move between skin cells. Larger particles, however, stay on the skin surface or within the hair follicles. They act like little storage spots, slowly releasing their special ingredients over time.

Many skin products also include ingredients that form a film on your skin when they dry. This film acts like a barrier. It protects your skin from foreign materials and also helps keep your skin hydrated by stopping water from evaporating.

Another way to protect your skin is by adding positively charged ingredients. These can change the natural charge of your skin and create an electrostatic field to protect it from pollutants. For example, U.S. Pat. No. 9,737,497 Issued to Ashok Wahi, et.al. in 2017 disclosed the use of charged ingredients in products applied near the nose to attract oppositely charged pollutants, thereby preventing them from being inhaled.

Beyond just protection, skin products aim for other benefits. These often include reducing the appearance of wrinkles, tightening the skin, making it look brighter and younger, and many other cosmetic improvements.

“Tech Neck” is a growing problem. It is also known as “Turkey Neck.” These terms refer to the early aging of the neck caused by looking down at electronic devices for long periods. The constant downward gaze strains the neck muscles and skin, leading to horizontal lines, sagging, and premature wrinkles. The repetitive motion of looking down often contracts neck muscles, which can break down collagen and elastin, leading to sagging and wrinkles. The skin on the neck and jawline is thinner and more delicate than facial skin. It has fewer oil glands, less collagen, and thinner support tissues, making it less resistant to constant movement, gravity, and repeated folding. When combined with long hours of looking down at screens, poor posture, and the natural loss of collagen, the result is a less defined jawline, horizontal neck lines, and an overall loss of firmness.

People are increasingly looking for skin products that have an immediate effect. They want to quickly smooth out wrinkles and fine lines and make signs of fatigue disappear, even if only for a short time. Such products use tensioning agents. A “tensioning agent” is an ingredient that can make the skin taut, reducing or even immediately making wrinkles and fine lines disappear. These agents are often natural or man-made polymers that are mixed in water. When applied, they can form a film that causes the top layer of the skin to tighten.

However, these tensioning polymer systems can sometimes make users feel uncomfortable, especially those with sensitive skin. Also, the tightening effect often doesn't last very long because the film formed on the skin tends to crack when you make facial expressions. This happens because these tensioning agents create a film on the skin that is quite stiff and not flexible.

To achieve many different effects from a single skin product, you might think of adding different ingredients, with each ingredient doing a specific job, for example, combining a film-forming ingredient with an anti-aging ingredient in one cosmetic product. However, this approach can become complicated and isn't easily done when the product has many ingredients and aims for multiple benefits. Some ingredients are incompatible with others, which can cause them to separate or break down. This becomes even more challenging when the skin product includes nanoparticles. It's very common for nanoparticles to be made separately and then require purification (cleaning) to remove specific materials before being added to the final product. This purification is usually done using methods like spinning them very fast (centrifugation), filtering, or osmosis. These techniques are typically long and expensive. So, choosing the right nanoparticle type and preparation method is crucial to allow the nanoparticles to be added to the skin product without needing these lengthy steps.

After looking closely at several products on the market, it becomes evident that current products do not usually achieve all the multiple effects needed for the skin, especially moisturizing, protection, and anti-aging, all in one. A single product usually only provides some benefits. Therefore, consumers often need to use more than one product. This also creates a risk of incompatibility between different products. For example, most moisturizers use hyaluronic acid as a key ingredient. However, our tests showed that hyaluronic acid is incompatible with cationic ingredients like Behentrimonium chloride, which other products use to protect skin from pollutants. Similarly, sorbic acid or sorbate, used as a preservative in some products, was found to be incompatible with Behentrimonium chloride.

Given the various issues affecting skin, such as environmental pollutants, signs of aging, water loss, and microbial infections, there is still a strong need for a skin product that works through different mechanisms or phases together to achieve the maximum benefits for the skin. There is also a need for a way to prepare nanoparticles and add them to the skin product without needing lengthy or costly steps.

### DISCUSSION OF THE PRIOR ART

U.S. Pat. No. 12,036,329, issued to Tzachev in 2024, entitled “Mucoadhesive Dispersion Nanoparticle System and Method for Production the Same,” teaches a dispersion nanoparticle system that can be used in pharmacy, medicine, and cosmetics, especially by administration to surfaces with intensive physiological clearance such as nasal, pulmonal, ocular, oral, throat, rectal, vaginal surfaces etc. While this reference discloses a dispersion nanoparticle system applicable to mucosal barriers, it does not teach a system for the skin or dermal barrier, which is recited in the invention of the present application. Mucosal barriers differ substantially from the skin or dermal barrier. For example, the skin has hair follicles that are not present in the mucosal barriers. Absorption from the skin differs from the mucosal barriers because of the difference in cellular structure.

U.S. Pat. No. 9,918,913, issued to Juliana Bucchi Alencastre Moroz, et.al., in 2018, entitled, “Nanostructured Conditioning Cosmetic Composition, the Use Thereof in Cosmetic Preparations, and a Conditioning Shampoo,” teaches conditioning cosmetic compositions comprising a nanostructured system formed by lipidic nanoparticles based on at least one from oil, butter, and wax, which encapsulate at least one cationic surfactant, as well as its use cosmetics. It also recites a conditioning shampoo comprising the cosmetic composition. Adjuvants that are usually employed in the cosmetic, hygiene and personal care industry may be included in the shampoo. These may include antioxidants, film-forming agents, moistening agents (humectants and emollients), preservers, thickeners, pH adjusters, sequestering agents (or chelating agents), fragrances or perfumes, cleansing agents and other cosmetically acceptable components. The composition may be used in shampoos, conditioners, hydrating masks, liquid or bar toilet soaps. While this reference teaches cationic nanoparticles and cosmetic compositions that include cationic nanoparticles and may include film forming ingredients, it is silent regarding a high concentration of a cationic ingredient, which is a key ingredient of the present invention. The high concentration of cationic ingredient in the present application confers a positive charge to the film that cannot be achieved using concentrations commonly used in the cosmetic formulations. In addition, this reference teaches compositions applied to the hair which differs substantially from the skin or dermal barrier. For example, the skin has hair follicles and live cell layers that are not present in the hair. Prime-function ingredients can be absorbed by the skin but not by the hair. Therefore, a formulation for the skin differs substantially from a formulation for the hair.

U.S. Pat. No. 6,428,814, issued to Bosch, et.al., in 2002, entitled “Bioadhesive Nanoparticulate Compositions Having Cationic Surface Stabilizers,” teaches a stable composition comprising (a) active agent particles and (b) adsorbed to the surface thereof at least one cationic surface stabilizer. The cationic surface stabilizers prevent aggregation of the nanoparticles and increase bioadhesion of the nanoparticles to biological substrates, such as an insect, teeth, bone, nails, chitin, feathers, scales, mucosa, skin, hair, plant tissue, etc. The particles may consist of pharmacologically active compounds (e.g., drug compounds for human or veterinary use), agricultural chemicals (pesticides, herbicides, fertilizers, and the like), cosmetic agents, consumer compositions (coloring agents, flavors, or fragrances), or other materials that function by interacting with biological substrates. In addition, the reference discusses methods of preparing and using such bioadhesive nanoparticulate compositions. The bioadhesive nanoparticulate compositions of this patent reference coat the targeted surface with a continuous and uniform film, invisible to the naked eye. The composition taught by this patent must include an active agent to perform the desired effect, while the dermal benefits of the present invention can be achieved without an active agent. In addition, this '814 patent reference does not teach a film forming ingredient that forms a film on the skin in a way completely different from the film formed by the bioadhesive nanoparticulate compositions. The film forming ingredient is a key component in the invention of present application. The skin protection barrier and moisturizing effect of the film formed by a film forming agent like Pullulan or Hydroxyethyl Cellulose is continuous and resistant to peeling or removal by touching, facial expression, and simple washing. The '814 patent is also silent regarding the presence of the high concentration of a cationic ingredient separate from the cationic nanoparticles, which is discussed in the present application.

U.S. Patent Application Publication No. US 2009/0246163, published on Oct. 1, 2009 on a patent application by Ashok Wahi, entitled, “Antistatic Skin Products and Method,” teaches a composition containing cationic agents that neutralize a negative charge in the skin, thereby reducing the distance between the pores. The composition claims to soften and condition the skin. The formulation described in this reference contains higher concentrations of cationic agents than other commercial products. This results in neutralizing the negative charge on the skin and relieving tension, softening and moisturizing the skin. Its formulation claims to condition dry, cracked, and chapped skin so as to retain moisture, and to condition hair. However, the composition described in this reference does not include cationic nanoparticles that penetrate into the pores, nor does it include a film forming feature that creates a protective coating that isolates the formulation on the skin from pollutants in the ambient air.

PCT International Application Publication No. WO 00/48555 on an application filed by Michael David Bell on Feb. 17, 2000, entitled, “Cosmetic Compositions,” teaches a skin conditioning formulation containing a cationic agent and a film forming polymer resistant to removal upon washing. The film forming polymer is said to enhance effective activity of the composition on the skin. Bell teaches cosmetic compositions that display good moisturization and in-use rub-in and absorption characteristics, as well as excellent skin feel, skin softness, and skin smoothness benefits. The cosmetic compositions are in the form of an emulsion of one or more oil phases in an aqueous continuous phase. However, Bell does not teach the use of cationic nanoparticles that penetrate deep into the pores, effectively enhancing the duration of cationic action even after the composition is washed away.

### SUMMARY OF THE INVENTION

The present invention comprises a method of treating a person's skin by applying a composition containing various active ingredients underneath a film that coats and protects the skin. The primary active ingredients adhere directly to the skin. They produce a cationic charge that creates a positively charged electrostatic field that surrounds the skin. It is well known that cationic ingredients can be used to soften and condition a person's skin. The composition described herein not only produces a cationic charge on the skin surface, but it also contains cationic nanoparticles that penetrate into the skin pores. These nanoparticles continue to provide a cationic charge to the skin for some time after the composition is removed by washing with soap and water. The cationic ingredients neutralize excess electrons that reside on the tiny hairs and the skin itself.

Various substances in the air that cause damage to the skin are negatively charged. Among these are dust, pollen, and various microorganisms. The positive charge created by applying the cationic composition attracts these harmful substances to the skin. However, instead of adhering directly to the skin, they adhere to a film that coats and protects the skin. The composition's active ingredients perform their prescribed functions in direct contact with the skin under the protective layer of the film.

The composition also contains active ingredients that promote anti-aging functionality. The composition's ingredients include sunscreens, along with those having antioxidant and anti-inflammatory properties. They also include those having materials that perform anti-aging, hydrating, and skin firming functions.

This invention also discloses and claims a novel skincare product designed for everyday use, combining three key components that work together to improve the skin's health and appearance. The first component is a positively charged (cationic) nanoparticle, usually made from solid lipids, that can carry active ingredients like Bakuchiol—a natural alternative to retinol. These nanoparticles are mixed into a skin-safe base that includes film-forming ingredients and other positively charged components, helping the product stick to the skin and enhance the absorption of prime-function (active) ingredients. The second component is a film-forming substance, preferably a natural polymer like Pullulan, which creates a thin, breathable layer on the skin that helps hydrate, tighten, and protect it while still allowing natural sweating. The third component is a positively charged ingredient such as Polyquaternium-22, which strengthens the film's grip on the skin, improves firmness, tightness, and acts as a defense against pollutants. Together, these three components create a product that adheres well to the skin, delivers active ingredients more effectively, and forms a protective, tightening layer in order to achieve instant and long-term skin benefits.

Finally, the invention discloses and claims a simplified manufacturing process that allows the nanoparticles to be made and used directly in the formula without the need for extra purification steps.

### OBJECTS OF THE INVENTION

It is an object of this invention to provide a method of treating a person's skin to soften and protect it

It is also an object of this invention to provide a composition for dermal treatment that includes multiple phases to achieve multiple dermal benefits. This composition includes cationic nanoparticle formulation as one of the phases. It also includes at least one film forming ingredient, at least one cationic ingredient and may include additional ingredients.

It is also an object of this invention to provide the method of preparing the cationic nanoparticle formulation and the method of preparing the final composition for dermal treatment.

### DETAILED DESCRIPTION OF THE INVENTION

One problem this invention solves is how to get beneficial ingredients into the skin effectively. Some ingredients that fight aging or protect from pollution need to stay on the skin longer to work well. Also, skin needs to be properly hydrated and its structure needs to be good so it can absorb these ingredients.

This invention provides a solution as a formulation applied to the skin using three main parts also referred as components or phases. First, cationic nanoparticles help these ingredients stay on the skin's surface and in hair openings, allowing for a higher concentration without causing harm. These nanoparticles also help the skin absorb other beneficial ingredients and can slowly release ingredients over time. Second, a film-forming ingredient like Pullulan creates a protective layer that holds in moisture and can contain beneficial ingredients. Third, a positively charged ingredient helps the film stick strongly to the skin and protect from pollutants. These three main components or phases of the composition of this invention work together to achieve the desired dermal benefits like anti-aging, protecting the skin from environmental pollution, moisturizing and rejuvenating it. The composition is formulated for daily or routine use by the consumer.

The First Phase/Component is the cationic nanoparticle formulation, preferred to be solid lipid nanoparticles (SLNs). These cationic nanoparticles are formulated to have a particle size larger than 10 nm, preferred to be larger than 100 nm and more preferred in the range of about 200 nm to about 800 nm. Nanoparticles smaller than 10 nm are suitable for deep penetration into the skin. The size of the cationic nanoparticles of this invention allows them to remain on the skin surface and within the hair follicles and prohibits their deep penetration into the skin. This feature allows the cationic ingredient that confers the positive charge to the nanoparticles to be loaded into the nanoparticles at an amount or concentration higher than what is usually added into a solution, without increasing the toxicity to the skin. The positive charge allows the nanoparticles to strongly adhere to the skin and protect it from the environmental pollutants by different means including generating an electrostatic field.

In another mechanism, the cationic nanoparticles also possess some occlusive properties. Following the evaporation of water from the dermal composition applied to the skin surface, the cationic nanoparticles together with the film forming ingredient form an adhesive layer occluding the skin surface. Thus, hydration of the stratum corneum increases, which by reducing corneocyte packing and widening of the inter-corneocytes gaps can facilitate prime-function ingredient penetration into deeper skin layers. In this mechanism, the cationic nanoparticles facilitate the absorption of other prime-function ingredients in the composition, whether the prime-function ingredient is encapsulated in the cationic nanoparticles or present in the composition outside the nanoparticles.

Another benefit of the cationic nanoparticles is the reduction of appearance of wrinkles and fine lines due to their optical and tightening effects. The optical effect comes from its color and easily filling the gaps in the skin. The tightening effect comes from the cationic charge, which enables the particles to firmly hold the skin around the fine lines and wrinkles.

The cationic nanoparticles in this invention provide the desired effects without containing any prime-function ingredient. These effects include tightening and moisturizing. They can also improve the absorption of prime-function ingredients, like Bakuchiol, present in the composition outside the cationic nanoparticles. On the other hand, prime-function ingredients like Bakuchiol can be loaded inside the cationic nanoparticles. In this case, the cationic nanoparticles enhance the absorption of the prime-function ingredient by acting as a depot that remains on the skin surface and within the hair follicles and releases the prime-unction ingredient over time. The sustained release of the prime-function ingredient reduces the side effects like the irritation associated with some prime-function ingredients.

The cationic nanoparticles were prepared in a method that is practical, low cost and does not require lengthy and costly purification. The ingredients in the cationic nanoparticle formulation became part of the final dermal composition. In one embodiment, cationic solid lipid nanoparticles are prepared using Cetyl Palmitate as the matrix lipid, Behentrimonium Chloride as the cationic ingredient and polysorbate 20 as the emulsifier. All these ingredients are compatible with dermal applications and there is no need for cationic nanoparticle purification before adding them to the final composition.

The Second Phase/Component in this invention is the film forming ingredient. The formulation contains one or more film forming ingredients, preferred to be a natural polymer, more preferred Pullulan. After drying, Pullulan produces a continuous film on the skin surface. This film protects the skin from pollutants and contributes to the moisture retention, keeping the skin hydrated.

Pullulan also has firming and tightening properties, both help to reduce the appearance of wrinkles and signs of aging. Furthermore, the film can be embedded with prime-function ingredients like Bakuchiol, which has anti-aging properties. The prime-function ingredient can be water soluble or water insoluble. The water-soluble ingredient is present in a solution form while the water-insoluble ingredient is present in an emulsion form. After applying the dermal composition, some water will evaporate, leaving Pullulan film in the form of continuous thin layer on the skin that gives a bright appearance. The film forming ingredient is preferred to be of essentially neutral charge to reduce the potential of incompatibility with the positively charged ingredients and cationic nanoparticles. The dermal composition may include more than one film forming ingredient, for example, Pullulan and Hydroxyethyl Cellulose (HEC). Some film forming ingredients may have more than one function. HEC for example works as film forming polymer and viscosity adjusting ingredient contributing to higher viscosity and more stable emulsion or suspension.

The film-forming agent in this product creates a lightweight, breathable layer on the skin. It uses water-attracting ingredients such as Pullulan and Hydroxyethyl Cellulose, which form a gel that holds a large amount of water. As this water slowly evaporates, it leaves behind tiny pores that are so small they can only be seen with special instruments. Because of these microscopic openings, the film still allows your skin to breathe and does not block sweat. The positively charged nanoparticles in the formula are also extremely small, much smaller than skin pores, so they do not cause any clogging even when they settle into hair follicles.

The film formed on the skin protects against airborne pollutants. Most of these pollutants have a negative electrostatic charge. These include dirt, mites, pollen, and various microorganisms. The cationic ingredients from the composition applied to the skin attract these pollutants to the skin. Without the film, these pollutants would come in close contact with the skin itself. The film serves as a protective layer forming a barrier allowing the active ingredients to treat the skin underneath the film while keeping the pollutants at bay. The pollutants do not penetrate the film.

The Third Phase/Component includes at least one cationic ingredient, such as Polyquaternium-22 in the dermal composition. This cationic ingredient confers a strong positive charge to the applied film, making it adhere strongly to the skin, improving the tightening and firming effects of the film, and increasing the protection against the environmental pollutants by multiple means including electrostatic field. The cationic ingredient is added to the film as a true solution or as a colloidal dispersion or a mixture of both, and it is either added to the aqueous phase or the oil phase or both, depending on its solubility in water or oil, molecular weight and structure.

After applying the product, the skin immediately feels smooth and soft. This silky feeling lasts for hours, even after washing. The effect comes from the way the positively charged ingredients in the formula temporarily balance the natural negative charge of the skin. Even once the product is removed, this gentle balancing effect stays for a while, leaving the skin feeling calm and supple.

The Three Phases/Components in this invention work together to achieve dermal benefits, keep the skin healthy, and improve skin appearance in multiple ways. The combined actions enhance the absorption of the ingredients that need to be absorbed like Bakuchiol. The longer the prime-function ingredient remains in contact with skin, the better it is absorbed. The cationic nanoparticles improve skin hydration and skin cell structure to facilitate the absorption. The film formed by the film forming ingredient like Pullulan also improves skin hydration and retention of the formulation on the skin. The positive charge of the formed film makes it adhere longer to the skin surface and resist peeling or being lost due to facial expressions, touching, scratching or simple washing.

The positive charge conferred by the cationic nanoparticles increases the overall positive charge of the film and improves its adherence to the skin. The cationic nanoparticles act as anchors to affix the film on the skin. As a result, improved protection against environmental pollutants is achieved. The firming and tightening effect of the cationic film is improved with the addition of cationic nanoparticles due to the increased adherence to the skin and the ability of the cationic nanoparticles to reach and remain inside the hair follicles and fine lines.

The presence of cationic nanoparticles in the film reduces the appearance of wrinkles and fine lines due to the tightening and firming effect and also due to the optical effect of the cationic nanoparticles which easily fill inside the fine lines.

In addition to its moisturizing and protective benefits, this formula also helps reduce the appearance of wrinkles and fine lines right away. This is because the film it forms gently tightens the skin, making lines look less noticeable or even disappear temporarily. At the same time, the product may include active ingredients such as Bakuchiol, which support skin renewal and collagen production. These longer-term benefits take a few weeks to develop, but with regular use, they continue to improve the look and feel of your skin over time.

The nanoparticles may include ceramides. Ceramides are neutral (non-ionic) lipids that can be found naturally in skin. They do not carry a charge, thus making them compatible with emulsifiers and surfactants in cosmetic formulations. In oil-in-water emulsions, they enhance the skin's barrier function by replenishing lipids, leading to improved moisture retention. They contribute to a smoother texture that enhances the sensory experience. Incorporating ceramides into nanoparticles can improve their stability and facilitate deeper skin penetration. Including them in nanoparticle emulsions can enhance the delivery of active ingredients, potentially increasing the efficacy of the formulation. Inclusion of ceramides in a cosmetic formulation benefits the skin by restoring the skin's barrier to prevent water loss. By restoring the lipid matrix, ceramides help maintain optimal skin hydration levels, reducing dryness and flakiness. The improved hydration and barrier function can diminish the appearance of fine lines and wrinkles, promoting a youthful complexion.

The cationic film supported with the cationic nanoparticles was found to withstand multiple simple and normal washing times and can only be removed with intentional washing stronger than normal. Therefore, the film becomes fixed to the skin and withstands washing, scratching, facial expressions, and other simple and normal removing factors. The presence of cationic nanoparticles surprisingly make the film have these properties which are not attained when the cationic ingredients are added to the dermal composition even at higher concentration. The positive charge is known to have cell toxicity; therefore, one cannot simply add more of the cationic ingredient to make the film more cationic. The presence of cationic nanoparticles was crucial to achieve the balance between efficacy and toxicity.

The formulation may also include a sunscreen to block ultraviolet rays that cause sun damage. It may also include antioxidants such as Vitamin C and E, which would reduce damage from free radicals that are harmful molecules that break down skin cells. It may further include moisturizers (to keep skin plumper and smoother) and peptides (to help signal skin to make more collagen). It May include exfoliants that remove dead skin so that new, fresher skin shows through.

We developed the method to prepare the dermal composition in a way to include the cationic nanoparticles with minimal manufacturing steps. The following tables provide different embodiments of the dermal composition and the preparation method.

The following three examples (REF-1, REF-2, and REF-3) are cosmetic compositions prepared without cationic nanoparticles. They are part of the formulation development and served later as references for comparison.

### Example Ref-1

| | |
|---|---|
| Ingredient | Percentage |

A

| | | |
|---|---|---|
| 1. | Purified Water | 40.0-50.0 |

B

| | | |
|---|---|---|
| 1. | Cellulose Gum | 0.2-0.7 |
| 2. | Sodium Silicate | 10.0-17.0 |
| 3. | PPG-3 Benzyl Ether | 3.0-7.0 |
| | Myristate | |
| 4. | Propylene Glycol | 3.0-5.0 |
| 5. | Glycerin | 0.5-3.0 |
| 6. | Sodium Coco PG- | 1.0-3.0 |
| | Dimonium Chloride | |
| | Phosphate | |
| 7. | Hyaluronic Acid | 0.1-0.5 |
| 8. | Phenoxyethanol | 0.5-0.8 |
| 9. | Potassium Sorbate | 0.2-0.5 |
| 10. | Aloe Vera 10x Concentrate | 0.5-2.0 |

The above formulation is prepared following common steps known to a cosmetic chemist.

### Example Ref-2

| | |
|---|---|
| Ingredient | Percentage |

A

| | | |
|---|---|---|
| 1. | Purified Water USP | 40.0-60.0 |
| 2. | Cellulose Gum | 0.5-2.0 |
| 3. | Magnesium Aluminum | 0.5-2.0 |
| | Silicate | |
| 4. | Hydroxyethyl Cellulose | 0.5-2.0 |
| 5. | Behentrimonium chloride | 3-7.0 |

B

| | | |
|---|---|---|
| 1. | Sodium Silicate | 17.0-25.0 |
| 2. | PPG-3 Benzyl Ether | 3.0-7.0 |
| | Myristate | |
| 3. | Propylene Glycol | 3.0-7.0 |
| 4. | Glycerin | 3.0-5.0 |
| 5. | Sodium Coco PG- | 1.0-3.0 |
| | Dimonium Chloride | |
| | Phosphate | |
| 6. | Hyaluronic Acid | 0.1-0.5 |
| 7. | Phenoxyethanol | 0.1-0.5 |
| 8. | Potassium Sorbate | 0.5-0.8 |
| 9. | Aloe Vera 10x Concentrate | 0.5-3.0 |

The above formulation is prepared following common steps known to a cosmetic chemist.

### Example Ref-3

| | |
|---|---|
| Ingredient | Percentage |

A

| | | |
|---|---|---|
| 1. | Purified Water USP | 50.0-70.0 |
| 2. | Cellulose Gum | 0.5-2.0 |
| 3. | Hydroxyethyl Cellulose | 0.5-2.0 |
| 4. | Behentrimonium chloride | 8.0-13.0 |
| 5. | Aloe Vera 10x Concentrate | 0.5-3.0 |

B

| | | |
|---|---|---|
| 1. | Glycerin | 3.0-7.0 |
| 2. | Pentylene Glycol | 3.0-7.0 |
| 3. | Hyaluronic Acid | 0.3-1.0 |
| 4. | Bakuchiol | 1.0-2.5 |
| 5. | Phenoxyethanol | 0.1-0.8 |
| 6. | Potassium Sorbate | 0.1-0.5 |
| 7. | Pullulan | 3.0-5.0 |
| 8. | Nannochloropsis Octulata | 3.0-5.0 |
| | Extract | |

The above formulation is prepared following common steps known to a cosmetic chemist.

The following examples provide cationic nanoparticle formulations together with final dermal compositions. A and B ingredients represent the cationic nanoparticle formulations.

TABLE 1

| | Ingredient | Percentage | Remark |
|---|---|---|---|
| A | | | |
| 1. | Behentrimonium | 5.0-15.0 | Cationic compound |
| | Chloride | | |
| 2. | Cetyl Palmitate | 10.0-15.0 | |
| 3. | Argan Oil | 5.0-8.0 | |
| 4. | Bakuchiol Oil | 1.0-2.0 | Anti-aging |
| B | | | |
| 1. | Purified Water | QS | |
| 2. | Polysorbate 20 | 2.0-4.0 | |
| C | | | |
| 1. | Dimethicone | 1.0-5.0 | |
| 2. | Phenoxyethanol and | 0.5-1.5 | |
| | Caprylyl Glycol | | |
| 3. | Hydroxyethyl cellulose | 0.1-1.0 | |
| 4. | Hydroxypropyl | 1.0-2.0 | |
| | Methylcellulose (HPMC) | | |
| 5. | Glycerin | 2.5-5.5 | |
| 6. | Propylene Glycol | 2.5-5.5 | |
| 7. | Pullulan | 3.5-5.5 | Film forming |
| 8. | Aloe Vera 10x | 2.0-5.0 | |
| | Concentrate | | |

### Method of Preparation

‘A’ ingredients were mixed and heated to melt then added to ‘B’ phase preheated to 60-70 deg. C. The mixture was mixed, then homogenized to make a hot emulsion, and then cooled down to room temperature to form the cationic nanoparticles.

‘C.’ ingredients except for C1 and C2 were added to the water and stirred until completely dissolved. C1 and C2 were then added and mixed to make a homogenous mixture, and then the cationic nanoparticle suspension was added and mixed to make the final homogenous composition. The pH was then adjusted to a suitable value, preferred 5.5-6.5 with lactic acid, citric acid, sodium citrate and sodium hydroxide.

TABLE 2

| | Ingredient | Percentage | Remark |
|---|---|---|---|
| A | | | |
| 1. | Behentrimonium Chloride | 0.3-3.0 | Cationic compound |
| 2. | Cetyl Palmitate | 10.0-15.0 | |
| 3. | Argan Oil | 1.0-3.0 | |
| 4. | Bakuchiol Oil | 1.8-2.5 | Anti-aging |
| B | | | |
| 1. | Purified Water | QS | |
| 2. | Polysorbate 20 | 3.0-4.0 | |
| C | | | |
| 1. | Polysorbate 80 | 3.0-5.0 | |
| 2. | Dimethicone | 3.0-5.0 | |
| 3. | Caprylic/capric triglyceride | 5.0-7.0 | |
| 4. | Phenoxyethanol and | 0.5-1.0 | |
| | Caprylyl Glycol | | |
| D | | | |
| 1. | Aloe Vera 10x Concentrate | 2.0-5.0 | |
| 2. | Polyquaternium-22 | 9.0-13.0 | Cationic compound |
| 3. | Pullulan | 1.5-9.0 | Film forming |
| 4. | Hydroxyethyl cellulose | 1.5-3.0 | |
| 5. | Xanthan Gum | 0.2-1.0 | |
| 6. | Glycerin | 2.0-5.0 | |
| 7. | Propylene Glycol | 3.0-5.0 | |

### Method of Preparation

‘A’ ingredients were mixed and heated to melt then added to ‘B’ phase preheated to 60-70 deg. C. The mixture was mixed then homogenized to make hot emulsion, and then cooled down to room temperature to form the cationic nanoparticles.

‘D’ ingredients were added to the water and stirred until completely dissolved. ‘C’ ingredients were mixed, then homogenized with ‘D’ ingredients to make an emulsion, and then the cationic nanoparticle suspension was added and mixed to make the final homogenous composition. The pH was then adjusted to a suitable value, preferred 5.5-6.5 with lactic acid, citric acid, sodium citrate and sodium hydroxide.

TABLE 3

| | Ingredient | Percentage | Remarks |
|---|---|---|---|
| A | | | |
| 1. | Behentrimonium Chloride | 3.5-5.0 | Cationic compound |
| 2. | Cetyl Palmitate | 10.0-20.0 | |
| B | | | |
| 1. | Purified Water | 25.0 | |
| 2. | Polysorbate 20 | 3.0-5.0 | |
| C | | | |
| 1. | Purified Water | QS | |
| 2. | Hydroxyethyl cellulose | 1.5-3.0 | |
| 3. | Glycerin | 2.0-5.0 | |
| 4. | Propylene Glycol | 3.0-5.0 | |
| 5. | Pullulan | 6.5-8.5 | Film forming |
| D | | | |
| 1. | Glyceryl Stearate (and) | 10.0-15.0 | |
| | PEG-100 Stearate | | |
| 2. | Bakuchiol Oil | 1.8-2.5 | Anti-aging |
| 3. | Argan Oil | 1.0-3.0 | |
| 4. | Caprylic/capric triglyceride | 2.0-5.0 | |
| E | | | |
| 1. | Phenoxyethanol and | 1.0-1.2 | |
| | Caprylyl Glycol | | |
| 2. | Aloe Vera 10x Concentrate | 2.0-5.0 | |
| 3. | Polyquaternium-22 | 3.5-7.5 | Cationic compound |

### Method of Preparation

‘A’ ingredients were mixed and heated to melt then added to ‘B’ phase preheated to 60-70 deg C. The mixture was mixed, then homogenized to make a hot emulsion, and then cooled down to room temperature to form the cationic nanoparticles.

‘C.’ ingredients were added to the water and stirred until completely dissolved. ‘D’ ingredients were melted, mixed, and then homogenized with preheated ‘C’ ingredients to make an emulsion. The emulsion was cooled below 45° C., and then ‘E’ ingredients and the cationic nanoparticle suspension were added and mixed to make the final homogenous composition. The pH was then adjusted to a suitable value, preferred 5.5-6.5 with lactic acid, citric acid, sodium citrate and sodium hydroxide.

TABLE 4

| | Ingredient | Percentage | Remarks |
|---|---|---|---|
| A | | | |
| 4. | Behentrimonium Chloride | 3.5-6.5 | Cationic compound |
| 5. | Cetyl Palmitate | 10.0-12.0 | |
| B | | | |
| 1. | Purified Water | 15 | |
| 2. | Polysorbate 20 | 3.0-5.0 | |
| C | | | |
| 1. | Purified Water | QS | |
| 2. | Hydroxyethyl cellulose | 1.5-2.5 | |
| 3. | Glycerin | 2.0-7.0 | |
| 4. | Propylene Glycol | 3.0-5.0 | |
| 5. | Pullulan | 1.5-3.5 | Film forming |
| D | | | |
| 1. | Glyceryl Stearate (and) | 7.0-11.0 | |
| | PEG-100 Stearate | | |
| 2. | Bakuchiol Oil | 1.8-2.5 | Anti-aging |
| 3. | Argan Oil | 0.8-1.2 | |
| 4. | Caprylic/capric triglyceride | 1.5-3.5 | |
| E | | | |
| 1. | Phenoxyethanol and | 1.0-1.5 | |
| | Caprylyl Glycol | | |
| 2. | Aloe Vera 10x | 2.0-3.0 | |
| | Concentrate | | |
| 3. | Polyquaternium-22 | 6.5-9.5 | Cationic compound |

### Method of Preparation

‘A’ ingredients were mixed and heated to melt, and then added to ‘B’ phase preheated to 60-70 deg C. The mixture was mixed, then homogenized to make hot emulsion and then cooled down to room temperature to form the cationic nanoparticles.

‘C.’ ingredients were added to the water and stirred until completely dissolved. ‘D’ ingredients were melted, mixed, and then homogenized with preheated ‘C’ ingredients to make an emulsion. The emulsion was cooled below 45° C., and then ‘E’ ingredients and the cationic nanoparticle suspension were added and mixed to make the final homogenous composition. The pH was then adjusted to a suitable value, preferred 5.5-6.5 with lactic acid, citric acid, sodium citrate and sodium hydroxide.

TABLE 5

| | Ingredient | Percentage | Remarks |
|---|---|---|---|
| A | | | |
| 1. | Cetyl Palmitate | 8.8-12.5 | |
| 2. | Behentrimonium Chloride | 3.0-5.0 | Cationic compound |
| B | | | |
| 1. | Purified Water | 20.0 | |
| 2. | Polysorbate 20 | 1.0-3.5 | |
| C | | | |
| 1. | Purified Water | QS | |
| 2. | Hydroxyethyl cellulose | 0.7-1.3 | |
| 3. | Glycerin | 2.0-6.0 | |
| 4. | Propylene Glycol | 3.0-8.0 | |
| 5. | Pullulan | 6.5-8.5 | Film forming |
| D | | | |
| 1. | Glyceryl Stearate (and) | 8.0-12.0 | |
| | PEG-100 Stearate | | |
| 2. | Bakuchiol Oil | 2.0-5.0 | Anti-aging |
| 3. | Argan Oil | 0.9-3.0 | |
| 4. | Caprylic/capric triglyceride | 0.6-3.0 | |
| E | | | |
| 1. | Phenoxyethanol and | 1.0-1.3 | |
| | Caprylyl Glycol | | |
| 2. | Aloe Vera 10x Concentrate | 2.0-5.0 | |
| 3. | Polyquaternium-22 | 7.0-13.0 | Cationic compound |
| 4. | Fragrance | 0.05-0.5 | |

### Method of Preparation

‘A’ ingredients were mixed and heated to melt, and then added to ‘B’ phase preheated to 60-70 deg. C. The mixture was mixed then homogenized to make a hot emulsion, and then cooled down to room temperature to form the cationic nanoparticles.

‘C.’ ingredients were added to the water and stirred until completely dissolved. ‘D’ ingredients were melted and mixed, and then homogenized with preheated ‘C’ ingredients to make an emulsion. The emulsion was cooled below 45° C. then ‘E’ ingredients and the cationic nanoparticle suspension was added and mixed to make the final homogenous composition. The pH was then adjusted to a suitable value, preferred 5.5-6.5 with lactic acid, citric acid, sodium citrate and sodium hydroxide.

The particle size of the cationic nanoparticles in the above embodiments ranges from 300 nm to 1500 nm. All of the formulations described in TABLES 1-5 representing various embodiments of the present invention operate in the manner that was disclosed herein. TABLE 5 represents the preferred embodiment. The same results may be achieved by varying the percentages for the prime-function and inactive ingredients. Varying the percentages for the prime-function ingredients affects the potency of the formulation. Varying the percentages for the inactive ingredients affects the consistency of the formulation. The desired results may be achieved by varying the ingredients and their amounts by those skilled in the art without undue experimentation.

The formulations taught in Tables 1-5 each represent a large number of embodiments. The ingredients are presented as percentages by weight and expressed in ranges. To prepare an individual composition, a chemist or formulator would use the recited ingredients, making certain to keep all ingredient percentages within the recited ranges for each ingredient. Doing so, would allow preparation of a product that would function as specified in this present application. However, a cosmetic chemist or formulator would normally adjust the percentages of these ingredients to achieve desired properties. This would involve some experimentation, but such experimentation would not be undue for those having ordinary skill in the art. As long as the ingredient percentages remain within the ranges presented in the above tables, formulations may be prepared that would have the desired properties that were discussed herein.

### Glossary

“Nanoparticles”, as used herein refer to a collection of particles, a substantial portion of which have diameters of less than about 5000 nm, such as less than about 4000 nm, less than about 2000 nm, less than about 1000 nm, from about 10 nm to about 800 nm, etc. The shapes of the particles are not particularly critical: spherical particles are typical. A substantial portion of the nanoparticles are deemed to have a specified diameter or a specified range of diameters when more than 50% have diameters of the specified diameter or within the specified range of diameters. Nanoparticles include but are not limited to: nanocapsule, nanosphere, nanoemulsion, dendrimers, solid lipid nanoparticle, nanostructured lipid carrier, polymeric nanoparticle, liposome, lipid nanoparticle, inorganic nanoparticle, and polymeric micelle.

“About”, as used herein includes plus/minus 30%, such as plus/minus 25%, plus/minus 20%, plus/minus 15%, plus/minus 10%, plus/minus 5%, and/or plus/minus 0% (to within the uncertainty of measurement methods).

“Prime-function” as used herein is the primary or principal role and purpose an entity, component or an element is designed to perform.

“Active Ingredient” as used herein is an ingredient of the formulation that conditions and/or treats the skin.

“Cationic Ingredient or Agent” as used herein is meant to refer to a substance in a formulation that carries a positive charge when it contacts with water at any pH, whether being in a solution, colloidal or suspension form. Examples include but not limited to quaternary ammonium compounds, amines, polymers with amino groups or quaternary ammonium groups and zwitterion compounds and polymers. It is also referred to as an active positively charged ingredient.

“Formulation” as used herein refers to a carefully engineered and developed product designed to interact with the skin in a specific way to deliver a desired outcome.

“Film Forming Ingredient” as used herein is an ingredient in the formulation that forms a continuous protective film on the skin.

“Pollutant” as used herein refers to solids, liquids, vapors, and gasses contained within the ambient air that cause harm to the person. They include pollen, dust, dirt, cigarette smoke, chemical gasses and vapors, and microorganisms. Generally, pollutants possess a negative electrostatic charge.

We claim:

1. A method for conditioning and treating a person's skin, wherein said skin comprises pores and hair follicles, and wherein the person resides in a place having ambient air that contains pollutants, said method comprising:
a) applying a formulation to the skin wherein the formulation comprises:
i) at least one active ingredient that conditions and treats the skin;
ii) a plurality of cationic nanoparticles that are small enough to penetrate into the pores, and wherein said nanoparticles contain the at least one active ingredient;
iii) at least one cationic agent that stays on the skin; and
iv) at least one film forming ingredient;
b) providing the skin with a positively charged electrostatic field;
c) forming a flexible protective film on the skin, wherein:
i) the film comprises first and second opposing surfaces wherein the first surface is closest to the skin, and the second surface is farther from the skin;
ii) the cationic nanoparticles, the active ingredients, and the cationic agent directly contact the skin and the first surface of the film;
iii) the second surface establishes a barrier that prevents penetration of the pollutants;
iv) said film is continuous with a plethora of microscopically small openings scattered throughout;
v) said openings are large enough to permit natural perspiration to exit through them; and
vi) said openings are small enough to block the pollutants from penetrating through them to the skin.

2. The method of claim 1, wherein the at least one active ingredient is selected from a group consisting of:
a) an active positively charged ingredient;
b) a nanoparticle matrix;
c) an anti-aging ingredient;
d) an anti-wrinkle ingredient;
e) an oil phase and emulsifier;
f) a skin tightening ingredient;
g) a soothing ingredient;
h) an anti-acne ingredient
i) an emollient;
j) a humectant;
k) a moisturizer;
l) a sunscreen;
m) a ceramide;
n) an anti-inflammatory; and
o) an antioxidant.

3. The method of claim 2, wherein the active positively charged ingredient is Behentrimonium Chloride.

4. The method of claim 2, wherein the nanoparticle matrix is Cetyl Palmitate.

5. The method of claim 2, wherein the anti-aging ingredient is Bakuchiol Oil or Argan Oil.

6. The method of claim 1, wherein the at least one film forming ingredient is selected from a group consisting of:
a) Pullulan;
b) Hydroxyethyl Cellulose;
c) Hydroxypropyl Cellulose; and
d) Xanthan Gum.

7. The method of claim 1, wherein the formulation further comprises a preservative.

8. The method of claim 1, wherein the formulation further comprises Sodium Silicate.

9. The method of claim 1, wherein the formulation further comprises Hyaluronic Acid.

10. A formulation for conditioning and treating a person's skin, wherein said skin comprises pores and hair follicles, and wherein the person resides in a place having ambient air that contains pollutants, said formulation comprising:
a) at least one active ingredient that conditions and treats the skin;
b) a plurality of cationic nanoparticles that are small enough to penetrate into the pores, and wherein said nanoparticles contain the at least one active ingredient;
c) at least one cationic agent present outside the nanoparticles; and
d) at least one film forming ingredient.

11. The formulation of claim 10, wherein the nanoparticle is composed of one or a combination of substances selected from a group consisting of:
a) solid lipid nanoparticles;
b) nanostructured lipid carriers;
c) liposomes;
d) polymeric nanoparticles;
e) dendrimers;
f) nanoparticle micelles; and
g) nanoemulsions.

12. The formulation of claim 10, wherein the at least one active ingredient is selected from a group consisting of:
a) an active positively charged ingredient;
b) a nanoparticle matrix;
c) an anti-aging ingredient;
d) an anti-wrinkle ingredient;
e) an oil phase and emulsifier;
f) a skin tightening ingredient;
g) a soothing ingredient;
h) an anti-acne ingredient
i) an emollient;
j) a humectant;
k) a moisturizer;
l) a sunscreen;
m) a ceramide;
n) an anti-inflammatory; and
o) an antioxidant.

13. The method of claim 12, wherein the nanoparticle matrix is Cetyl Palmitate.

14. The method of claim 12, wherein the anti-aging ingredient is Bakuchiol Oil or Argan Oil.

15. The formulation of claim 10, wherein the at least one cationic agent is selected from a group consisting of:
a) quaternary ammonium compounds;
b) polyquaternary ammonium compounds; and
c) cationic polymers.

16. The formulation of claim 15, wherein the at least one cationic agent is Behentrimonium Chloride or Polyquaternum-22.

17. The formulation of claim 10, wherein the at least one nanoparticle has a particle size in a range of between 10 nanometers and 5,000 nanometers.

18. The formulation of claim 10, wherein the at least one film forming ingredient is a natural polymer, a synthetic polymer, or a semi-synthetic polymer.

19. The formulation of claim 18, wherein the at least one film forming ingredient is selected from a group consisting of Pullulan, Hydroxyethyl Cellulose, Hydroxypropyl Cellulose, and Xanthan Gum.

20. The formulation of claim 10, wherein the at the least one active ingredient is present outside of the plurality of nanoparticles.

21. A method of preparing a skin conditioning formulation comprising a first group of ingredients, a second group of ingredients, a third group of ingredients, and a fourth group of ingredients, wherein:
the first group comprises Behentrimonium Chloride, Cetyl Palmitate, Argan Oil, and Bukuchiol Oil;
the second group comprises purified water and Polysorbate 20; and
the third group comprises Dimethicone, Phenoxyethanol; and
Caprylyl Glycol;
the fourth group comprises Hydroxyethyl Cellulose, Hydroxypropyl Methylcellulose, Glycerin, Propylene Glycol, Pullulan, and Aloe Vera 10× Concentrate;
said method comprising:
a) Mixing the ingredients of the first group together to form a first mixture;
b) heating and melting the first mixture;
c) heating the ingredients of the second group to between 60° C. and 70° C.;
d) adding the first mixture to the second group to form a second mixture;
e) homogenizing the second mixture;
f) cooling the second mixture to room temperature to form a cationic nanoparticle suspension;
g) completely dissolving the fourth group of ingredients in purified water to make a solution;
h) adding the third group of ingredients to the solution to form a homogenous mixture;
i) adding the cationic nanoparticle suspension to the homogenous mixture to form the final homogenous composition;
j) adjusting the pH of the final homogenous composition to between 5.5 and 6.5 by adding one or more ingredients selected from the group consisting of lactic acid, citric acid, sodium citrate, and sodium hydroxide.

22. The product produced by the method of claim 21.

23. A method of preparing a skin conditioning formulation comprising a first group of ingredients, a second group of ingredients, a third group of ingredients, and a fourth group of ingredients, wherein:
the first group comprises Behentrimonium Chloride, Cetyl Palmitate, Argan Oil, and Bukuchiol Oil;
the second group comprises purified water and Polysorbate 20;
the third group comprises Polysorbate 80, Dimethicone, Caprylic/Capric Triglyceride, Phenoxyethanol, and Caprylyl Glycol; and
the fourth group comprises Aloe Vera 10× Concentrate, Polyquaternium 22, Pullulan, Hydroxyethyl Cellulose, Xanthan Gum, Glycerin, and Propylene Glycol;
said method comprising:
a) Mixing the ingredients of the first group together to form a first mixture;
b) heating and melting the first mixture;
c) heating the ingredients of the second group to between 60° C. and 70° C.;
d) adding the first mixture to the second group to form a hot emulsion;
e) homogenizing the hot emulsion to form a cationic nanoparticle suspension;
f) completely dissolving the fourth group of ingredients in purified water to make a solution;
g) mixing the ingredients of the ingredients of the third group together to form a second mixture;
h) adding the second mixture to the solution to form a second emulsion;
i) homogenizing the second emulsion;
j) adding the cationic nanoparticle suspension to the second emulsion and mixing to form the final homogenous composition;
k) adjusting the pH of the final homogenous composition to between 5.5 and 6.5 by adding one or more ingredients selected from the group consisting of lactic acid, citric acid, sodium citrate, and sodium hydroxide.

24. The product produced by the method of claim 23.

25. A method of preparing a skin conditioning formulation comprising a first group of ingredients, a second group of ingredients, and a third group of ingredients, wherein:
the first group comprises Behentrimonium Chloride and Cetyl Palmitate;
the second group comprises purified water and Polysorbate 20;
the third group comprises purified water, Hydroxyethyl Cellulose, Glycerin, Propylene Glycol, and Pullulan;
said method comprising:
a) mixing the ingredients in the first group together;
b) heating the ingredients in the first group to melting;
c) preheating the ingredients in the second group to between 60° C. and 70° C.;
d) adding the ingredients of the first group to the ingredients of the second group to form a first emulsion;
e) homogenizing the first emulsion;
f) cooling the first emulsion to room temperature to form the cationic nanoparticles;
g) mixing the ingredients of the third group and stirring until they are completely dissolved in the water;
h) adding the cationic nanoparticles to the second emulsion to form a final composition.

26. The product produced by the method of claim 25.

27. A method of preparing a skin conditioning formulation comprising a first group of ingredients, a second group of ingredients, a third group of ingredients, a fourth group of ingredients, and a fifth group of ingredients, wherein:
the first group comprises Behentrimonium Chloride and Cetyl Palmitate;
the second group comprises purified water and Polysorbate 20;
the third group comprises purified water, Hydroxyethyl Cellulose, and Pullulan;
the fourth group comprises Glyceryl Stearate, PEG-100 Stearate, Bakuchiol Oil, Argan Oil, and Caprylic/Capric Triglyceride; and
the fifth group comprises Phenoxyethanol, Caprylyl Glycol, Aloe Vera 10× Concentrate, and Polyquaternum 22;
said method comprising:
a) mixing the ingredients in the first group together;
b) heating the ingredients in the first group to melting;
c) preheating the ingredients in the second group to between 60° C. and 70° C.;
d) adding the ingredients of the first group to the ingredients of the second group to form a first emulsion;
e) homogenizing the first emulsion;
f) cooling the first emulsion to room temperature to form the cationic nanoparticles;
g) mixing the ingredients of the third group and stirring until they are completely dissolved in the water;
h) melting the ingredients of the fourth group, adding them to ingredients of the third group to form a second emulsion.
i) cooling the second emulsion to about 45° C.;
j) adding the cationic nanoparticles to the second emulsion to form a final composition.

28. The method of claim 27, wherein the fifth group further comprises a fragrance ingredient.

29. The product produced by the method of claim 27.

30. The product produced by the method of claim 28.
